(19) United States
(12) Patent Application Publication
(10) Pub. No.: US 2010/0209300 A1
(43) Pub. Date: Aug. 19, 2010

(54) ANALYSIS SYSTEM WITH A REMOTE ANALYSING UNIT

(75) Inventors: Holger Dirac, Birkeroed (DK); Karsten Dupont Nielsen, Koebenhavn (DK); Jim Radmer, Frendensborg (DK); Jesper Peter Windum, Hilleroed (DK); Thorkild Ahm, Alleroed (DK)

Correspondence Address: MCCORMICK, PAULDING & HUBER LLP, CITY PLACE II, 185 ASYLUM STREET, HARTFORD, CT, 06103, US

(73) Assignee: Diramo A/S, Nordborg (DK)

(21) Appl. No.: 12/524,032

(22) PCT Filed: Jan. 25, 2008
(86) PCT No.: PCT/DK2008/000033

§ 371 Date: Apr. 29, 2010

(30) Foreign Application Priority Data
Jan. 26, 2007 (DK) PA 2007 00 126

(51) Int. Cl. G01N 21/00; G01N 33/00

(52) U.S. Cl. 422/82.05; 422/68.1

(57) ABSTRACT

A system for analysing a fluid, the system comprising a base station, an analysing unit being remote from the base station, and a substance collecting device being remote both from the base station and from the analysing unit, a first fluid communication link for communicating fluid between the base station and the analysing unit, and a second fluid communication link for communicating fluid between the analysing unit and the substance collecting device, wherein the analysing unit comprises sensing means adapted to provide data representing a content of a substance in the fluid and wherein the base station comprises data processing means being adapted to process the data to provide information regarding the content of the substance in the fluid.

### CROSS REFERENCE TO RELATED APPLICATIONS

This application is entitled to the benefit of and incorporates by reference essential subject matter disclosed in International Patent Application No. PCT/DK2008/000033 filed on Jan. 25, 2008 and Danish Patent Application No. PA 2007 00 126 filed Jan. 26, 2007.

### TECHNICAL FIELD

The present invention relates to a system for analyzing a fluid, and in particular to determine a content of a substance in the fluid. The system comprises an analyzing unit and a substance collecting device.

### BACKGROUND OF THE INVENTION

Systems of the above-mentioned kind can be used to measure concentrations of a substance in fluid, e.g. to measure a substance in a body fluid, e.g. to measure glucose.

The importance of being able to accurately and continuously measure concentrations of substance within substance such as tissue or some fluid like bodily fluid is well known in the field of medical art or science, like surveillance of a chemical process. One important medical application is to monitor the concentration of a chemical in biological environments, like monitoring glucose levels in the blood.

For patients suffering from diabetes it is often vital to monitor the levels of glucose, since it is known that elevated levels of glucose in the blood are indicative of conditions such as hyperglycemia and glycosuria resulting from inadequate production or utilization of insulin. Alternatively, abnormally low glucose concentrations may be an indication of overproduction of insulin. Therefore measurement of blood glucose concentration is an important tool for diagnosing, treating or controlling a variety of disorders in which the glucose concentration is known to be an indicator of the existence or severity of the condition. Situations thus exist in which the amount of insulin present is either in excess of or less than that required to handle the specific blood glucose level at any given time. Such situations are especially severe when an individual with a diabetic condition is under stress conditions, such as surgery or during childbirth.

Not only diabetics, but also non-diabetic patients may have the need of having a surveillance of their blood glucose level, like acutely ill patients treated with a pharmacologic dose of corticosteroid.

Within biotechnology other interesting applications are to maintain and control specific concentration levels of nutrients, such as glucose, in cell culture reactors, where a long-term stability is needed in order to provide feedback information required to control computerized delivery systems so that a particular chemical can be maintained within preset limits.

Examples of measuring the concentration of a specific chemical, such as glucose, in a solution is described in a number of documents, such as WO9939629A1 and U.S. Pat. No. 4,452,887. The latter describes a determination method where a test material or the reaction product thereof is oxidized using an oxidase enzyme, and hydrogen peroxide formed simultaneously with the oxidation is determined by various means. This has recently become important. The reason for this is that the determination of hydrogen peroxide can be accurately performed by a colorimeteric determination after a dye-forming reaction using peroxidase or by means of an electrode reaction. According to U.S. Pat. No. 4,452,887 a colorimeteric method based on the foregoing principle using a Trinder reagent is well known. In this method, hydrogen peroxide formed by the action of an oxidase enzyme is reacted with peroxidase to catalyze the oxidative coupling reaction of aminoantipyrine and a phenol and the dye thus formed is colorimetrically determined. The merit of the reaction system is that the same detection system can be utilized for different kinds of oxidase enzymes and the application of the system to various kinds of analysis is being investigated. Among these oxidase enzymes, particularly important enzymes in clinical chemistry are glucose oxidase, cholesterol oxidase, uricase, glycerol oxidase, phosphoglucose oxidase, etc.

In order to improve the systems enhancers may be introduced such as described in WO9105872A1, describing an enhanced chemiluminescent assay, in which a dihydrophthalazinedione such as luminol, a peroxidase such as HRP and an oxidant such as H2O2 are co-reacted in the presence of an enhancer such as (p)-iodophenol. The enhancer is generated by enzyme-catalysed reaction of a pro-enhancer, e.g. (p)-iodophenol phosphate is cleaved by alkaline phosphatase, enabling this enzyme to be assayed instead of peroxidase. Alternatively, the enhancer is added, an anti-enhancer such as (p)-nitrophenol is generated by enzymatic reaction of a pro-anti-enhancer such as (p)-nitrophenol phosphate and the reduction in luminescent emission is measured.

The chemi luminescent assays are described as “enhanced” in the sense that the total light emission of the reaction and/or the signal/background ratio is larger than that obtained in the same reaction carried out in the absence of an enhancer.

General patient monitoring systems are well known, as disclosed in U.S. Pat. No. 3,972,320 describing such a monitoring system producing an alarm at a central station when a monitored condition at a monitor station deviates beyond a predetermined limit. The monitor system is especially adapted for monitoring a vital function of plural patients in a hospital so that a single attendant is alerted if any patient needs emergency treatment. The monitor unit is portable by the patient, suitably in the form of a wrist-unit, and a communications link, suitably by radio frequency transmission, is provided for one-way transmission from the monitor station to the central station. Each monitor station develops and processes data to determine whether the monitored condition has a value exceeding a predetermined limit; if so, an identification signal is transmitted to the central station to signify that an emergency exists at that monitor station. Each monitor station includes a programmed data processor to eliminate the need for transmitting variable data to the central station. Only fixed or stored data is transmitted for the purpose of identifying the monitor station. The processor electronics is suitably implemented in large scale integrated circuitry.

A number of systems has been developed for such continuous measurement of substance, like the document WO 99/39629 describing an implantable sensing arrangement having long-term stability. The sensing arrangement utilizes microdialysis sampling techniques and includes a micro-flow reservoir having a reagent which reacts with a target chemical and a sensor connected to the micro-flow reservoir for detecting the reaction of the reagent and the target chemical. The sensor may include a thermopile or optical cell.

In one sensing arrangement, the invention includes (i) an optical cell and (ii) microdialysis tubing. This sensing arrangement combines microdialysis sampling techniques with the use of a microflow system employing an optical cell to create a system that can accurately measure the concentration of glucose and other chemicals in complex solutions bearing proteins. In this embodiment described in the document, the biochemical sensing system includes a pressurized container which includes collapsible bags for holding reagents, a calibration solution, and a sweep solution. These are regulated in their flow by resistance tubing, as hereinbefore described, whose diameter and length can been selected to achieve flow rates typically in the sub-microliter per minute regime.

The sweep solution is introduced by connecting tubing, typically microbore tubing, to a microdialysis fiber that is in diffusive contact with the test environment, e.g., a bioreactor perfusion loop. At flow rates of approximately 300 nl/min. and a retention time of about 2 minutes through a microdialysis fiber of about 10 to 2000 mm. long, the target-chemical concentration in the sweep fluid can reach diffusive equilibrium with the test environment. The return dialysate (i.e., sweep fluid containing the target-chemical) is then mixed with the particular reagent. The mixed solutions move down a single tube or capillary where the chemical reaction of the reagent with glucose proceeds and the optical change occurs, i.e., the reagent-dialysate mixing volume. The absorbance of the flow stream at the specific color of a chemically sensitive dye is measured by an optical cell having a light emitting diode and miniature diode photodetector. The resulting photodetector signal is calibrated in terms of glucose concentration by the microcontroller.

The microdialysis tubing, also referred to as a membrane hollow fiber, in contact with the test solution test is made from a material which is permeable to glucose but excludes large molecular weight materials. Typically, the microdialysis tubing is made of materials such as cellulose acetate, polysulfone, and polyacrylonitrile, usually in the form of hollow tubes in the order of 200 microns in diameter. The reagents that are mixed with the sweep fluid are chosen so that their colour or fluorescence change has a specific response to the biochemical desired, as is well known in the art.

An optical cell at the receiving end of the mixed reagent flow stream measures colour or fluorescence change, and the signal obtained therefrom is related to chemical concentration by microcontroller.

The micro-flow reagent reservoir may be remote from the sensor and connected to it by a catheter containing microbore tubing. This system may desirably take the form of a small storage reservoir which contains a means for refilling it in the reservoir. A typical system is illustrated as a reservoir containing the enzyme or chemical system being remote in location from the sensor and connected to it by a catheter of suitable length containing the microbore tubing and electrical lead wires. This system may desirably take the form of a small storage reservoir, perhaps of the size and shape of a pacemaker, which contains a means for refilling by way of a syringe needle through septa in the containment. This system may be refilled in a way analogous to implantable drug delivery systems whereby the septa is penetrated by a syringe needle through the skin.

This system however is not very suitable when a number of reagent fluids are mixed to the sweep fluid, or sample fluid. This is especially the case if a first fluid needs to have mixed sufficiently with the sweep fluid, before a second reaction fluid is added. The reason is that a connecting tube would be needed for the sweeping fluid and each of the different reagent fluids, and further tubes would be needed after each of the mixings, to give the reactions time to complete before news reagent fluids are added. This would require a number of connections of the different tubes, thereby enhancing the number of manufacturing steps, and the possibilities of harming one of the small and relatively fragile tubes. Further, given the micro dimensions of the tubes, it may be difficult to align them correctly and smoothly, so that the fluids to be mixed are laminated and mixed in a determined laminar and engineered manner.

Other methods of communicating flows in the order of micro litres pr minute comprise micro-channels formed in silicon or glass for chemical analysis. An example is a system for flow injection analyzes described in U.S. Pat. No. 5,644,395 where small quantities of chemical reagents and sample are intermixed and reacted within such a flow system, where the dimensions ensure capillary flow, and the reaction products are detected optically, electrochemically, or by other means. To regulate the flows micro-valves are mounted on the surface. The capillary channels comprise a section for mixing of the fluids, a section for the needed reactions to occur and a detection section.

It is also known to use the technique of analyzing by chemical reaction in the field of micro-dialysis for continuously monitoring the concentration of substance like glucose in tissue. In U.S. Pat. No. 5,640,954 a micro-dialysis probe is implanted in tissue and fed by a perfusion fluid that is removed as sample after enrichment with the substance from the tissue. The fluids are led through a tube system, where an enzyme is added and an electrochemical sensor registers a measurable chemical reaction. The flow rates in the system are quite small being in the range from 0.1 to 15 micro litres pr. minutes. To produce the flows a first and a second transport means are introduced, preferably in the form of rolling or piston pumps, where a compact set-up would be to use a single pump and control the flow rates by using tubes with different diameters.

In another patent, U.S. Pat. No. 6,572,566, the idea of having flows in channels is combined with direct analysis of a body fluid. The systems contain integrated reservoirs connected to the channels and an exchange region through which the substances from surrounding body fluids can be taken up into the channel, e.g. through a dialysis membrane. To propagate the fluids a pumping system is suggested based on a pressure container filled with a pressurized gas being in contact with a second container split in two parts by a flexible member. The first part contains a liquid and the second part receives the pressurized gas, displacing the flexible member and squeezing liquid into a channel system. A flow restrictor is located downstream of the pumping system to limit the amount of liquid emerging from the reservoir and to keep the flow constant.

A document WO2005111629 describes a micro-analysis system for analysing species with a fluid medium, said system containing sensing means for collecting species from the medium, said sensing means having an inlet and an outlet, analysing means comprising a channel, said channel defining at least one part for mixing, at least one part for reacting and at least one part for measuring detecting arrangement for determining the concentration of species at said part for measuring, a first fluid reservoir holding carrier fluid and at least one second fluid reservoir holding reagent fluid, connecting means, comprising first connecting means for fluid connection between the first fluid reservoir and the analysing means, second connecting means for fluid connection between the second fluid reservoir and the inlet of the sensing means and third connecting means for fluid connection between the outlet of the sensing means and the analysing means, said first connecting means comprising at least one first flow restricting means and said second connecting means comprising at least one second flow restricting means characterised by said micro-analysis system further comprising storage means containing said first fluid reservoir and said second fluid reservoir, said storage means being in downstream flow connection with means for pressurizing said fluid reservoirs, said storage means and said pressurizing means being separated from said analysing means.

In this document however, the analysing means (50) are an integral part of the analysis system (200) also comprising the reservoirs (12-15) and pressurizing means (1). Therefore, given e.g. that the system is operating to surveille a physiological condition of a patient, this system fails to combine the comfort for the patient not having to wear the whole analysis system (200), short response times by having short tubes connecting the sampling means (60) to the analysis system (200), and the reduced movability for the patient such short tubes would lead to.

### SUMMARY OF THE INVENTION

It is an object of this invention to improve the above described systems, and to introduce a system where a substance in a fluid continuously is measured having a cheep and easily exchangeable single-use part, that is easy to manufacture.

This invention thus concerns a system for analyzing a fluid, the system comprising a preferable reusable base station, an preferable cheep single-use analyzing unit being remote from the base station, and a preferable cheep single-use substance collecting device being remote both from the base station and from the analyzing unit, a first fluid communication link for communicating fluid between the base station and the analyzing unit, and a second fluid communication link for communicating fluid between the analyzing unit and the substance collecting device, wherein the analyzing unit comprises sensing means adapted to provide data representing the content of a substance in the fluid and wherein the base station comprises data processing means being adapted to process the data to provide information regarding the content of the substance in the fluid.

The first fluid communication link has smaller flow resistance than the second fluid communication link, and in order to ensure short response times of samples of the substance collected by the substance collecting device, the second fluid communication link is shorter than the first fluid communication link, this also being the argument for introducing the remote positioned analyzing unit.

The base station comprises a computing device, and a cheep single-use reservoir system, the reservoir system being connected to the analyzing unit by the first fluid communication link, and where and the reservoir system is detachable from the computing device. The base station further comprises pumping means being detachable from the reservoir system and thereby from the analyzing unit. This separation of a reusable base station from the single-use reservoir system, analyzing unit and substance collecting devise, together called the wet parts, makes it easy to exchange exhausted wet parts with new ones contained in a sterile package.

To transfer data and/or energy, the analyzing unit is in electrically communication with the computing device.

To ensure that no external substances pollutes the fluids in the reservoir system, the reservoir system comprises at least one flexible reservoir in pressure communication with a pressure chamber, the pumping means filling the pressure chamber with air or fluid, thereby squeezing the fluids out of the reservoir system, without ever being directly in contact with the fluids.

The measurement is based on adding fluids to the sample fluid to create an optical change representative of the concentration of the substance under investigation, therefore the analyzing unit comprises an optical sensor arranged in optical communication with a microfluidic chip.

Preferably at least the analyzing unit and the substance collecting device are made entirely of material(s) compatible to a MRI scan.

The analyzing unit is for analyzing the content of a substance in a fluid and therefore comprises sensing means adapted to provide data representing the content of the substance in the fluid, wherein the sensing means comprises,

an analysis microfluidic chip with at least one analysis channel and

an optical sensor.

The reactions giving the optical change are performed in the analysis microfluidic chip, and in order to ensure sufficient mixing of fluids at least one analysis channel of the analysis microfluidic chip comprises a meandering section.

To ensure that the optical sensor may observe the optical changes at least a section of the at least one analysis channel is covered by a transparent top part.

To distribute the fluids to the analysis microfluidic chip it is in fluid communication to at least one manifold chip channel of a manifold microfluidic chip.

To ensure that no undesired elements or particles enter the analysis microfluidic chip, then a filter is arranged between the manifold microfluidic chip and the analysis microfluidic chip.

To regulate the flows of the fluids in the system, at least one of the analysis microfluidic chip and the manifold microfluidic chip comprises at least one channel with a channel portion providing an increased flow resistance.

The increased flow resistance is preferable provided by a piece of capillary tube which is arranged in the channel.

Since the measurement is based on optical detection, it is important to ensure that no external light sources enters the analyzing unit, therefore a support structure is arranged between the optical sensor and the analysis microfluidic chip, the support structure comprising a first window arranged to facilitate optical communication between the optical sensor and the analysis microfluidic chip.

To ensure stability the optical sensor is arranged in a deepening provided in a first side of the support structure and the optical sensor is fixed in a casing comprising a second window, the casing and sensor is positioned in the deepening so that the first window aligns with the second window.

To ensure a free optical communication between the analysis microfluidic chip and optical sensor, and to ensure sufficient time for the reactions to give the optical detectable effect, the analysis microfluidic chip comprises a transparent top part covering at least one of the meandering sections, the top part being aligned with the first window.

In order to have a reservoir easy to construct, it comprises:
a pressure side element with an inner surface forming openings into a first group of cavities,
a fluid side element with an inner surface forming openings into a second group of cavities, and
a flexible or deformable membrane,
   the pressure side element and the fluid side element being arranged with opposing inner surfaces on opposite sides of the membrane so that openings into the cavities on one side of the membrane is aligned with openings into cavities on the opposite side of the membrane and so that the membrane separates the cavities on one side of the membrane from the cavities on the opposite side of the membrane, the inner surface of the pressure side element comprising a first pattern of recesses which in combination with the membrane forms fluid communication between the cavities of the first group of cavities and an external site.

To ensure a fluid outlet for the fluids in the reservoir the inner surface of the fluid side element comprises a second pattern of recesses which in combination with the membrane forms fluid communication between the cavities of the second group of cavities and an external site. Further to ensure that the membrane never blocks the fluid outlet, at least one of the first and second patterns of recesses extends into the cavities.

To form channels from the first and second patterns of recesses, at least one of the elements is bonded to the membrane, preferable by welding, such as laser welding or ultrasonic welding.

To ensure that a pressure gradient across the membrane may squeeze the fluids out of the reservoir, the membrane comprises in a first embodiment flexible portions being more elastic than a remainder portion of the membrane, the flexible portions being located between aligned cavities, and where the flexible portions are adapted to be deformed essentially into a shape of at least one of the two cavities located on each side of the flexible portion.

In a second embodiment the membrane sections are being shaped to fit at least roughly into an internal shape of at least one of two aligned cavities.

To seal the cavities of the fluid side element, the membrane is attached to the fluid side element around rim portions of the second group of cavities and around recesses of the second pattern of recesses.

In the same way, to seal the cavities of the pressure side element, the membrane is attached to the pressure side element around rim portions of the first group of cavities and around recesses of the first pattern of recesses.

To prevent flows of the fluids prior to use, the fluid side element comprises valve through holes, where the membrane is being connected to the fluid side element around pairs of the valve through holes, so that each valve through hole together with at least one other valve through hole on the inner surface of the fluid side element is surrounded by a joint zone along which the membrane and the fluid side element is connected. Then a removable valve member is positioned with a first end positioned on the flexible membrane to press the flexible membrane onto the fluid side element, thereby blocking any fluidic access across the valve through holes.

To make fluid communication between the two valve through holes of each pair of valve through holes, the fluid side element is connected to a manifold element having manifold recesses forming an internal geometry in an inner surface of the manifold element. The manifold element is attached to the fluid side element with the inner surface towards an outer surface of the fluid side element, the outer surface being opposite the inner surface of the fluid side element.

The second pattern of recesses is in fluid communication with the manifold recesses by reservoir through holes, the manifold recesses thereby making fluid communication between the second pattern of recesses and the valve through holes.

In order to safely store waste fluids returning from the analyzing unit, a waste chamber is introduced in the reservoir being in fluid communication with a first section of a recess formed in the inner surface of the fluid side element, the first section of the recess being in fluid communication with a first check valve through hole, and a second section of a recess being in fluid communication with a second check valve through hole, where each of the check valve through holes are in fluid communication with the internal of a check valve geometry of the manifold element were a check valve is arranged, insuring that fluid communication between the first and second check valve through holes is via the check valve.

The waste chamber is formed at least partly by a waste cavity provided in the inner surface of the fluid side element.

To ensure that no mixing of fluids occur in the manifold recesses if the connection of the manifold element to the fluid side element is not fluid tight, an drain channel is arranged between any two neighbouring manifold recesses, the drain channel being parallel to any two neighbouring manifold recesses.

To ensure an easy and quick attachable and detachable connection between the pumping means and the reservoir system, a connection is introduced with a male connector, a female connector, and a sealing member, the sealing member being fixed in a groove in the male connector, the female connector forming a cavity to receive the male connector and the sealing member being slightly larger than the cavity so that the sealing member engages an inner wall of the cavity when the male connector is received in the female connector.

### BRIEF DESCRIPTION OF DRAWINGS

The above and other features and advantages of the present invention will become more apparent by describing in detail exemplary embodiments thereof with reference to the attached drawings in which:

FIG. 1 shows the general system.

FIG. 2 shows a general micro-dialysis probe.

FIGS. 3A & 3B show a general microfluidic chip.

FIGS. 4A & 4B show tubes with a fluidic connection to a channel in a microfluidic chip.

FIG. 5 shows a reservoir system comprising the fluids.

FIG. 6 shows the bottom of the reservoir system and a manifold element.

FIG. 7 shows a simple illustration of the fluid reservoirs.

FIGS. 8A-8C show a fluid reservoir, empty, full and during operation of the system.

FIGS. 9A & 9B show two versions of the waste chamber.

FIG. 10 shows a diagram of the base station.

FIG. 11 shows the feature of drain channels.

FIG. 12 shows the pump connection.

FIG. 13 shows the analyzing unit system.

FIG. 14 shows an analysis microfluidic chip.

FIG. 15 shows a manifold microfluidic chip.

FIG. 16 shows a filter in a filter recess.

FIG. 17 shows a restriction collection.

FIG. 18 shows a flow restrictor inserted into a channel.

FIGS. 19A & 19B show an alternative to the manifiold element of the reservoir system.

FIGS. 20A & 20B show an alternative to the valve.

### DETAILED DESCRIPTION OF THE PREFERRED EMBODIMENTS

The following is a detailed description of the preferred embodiments of the invention.

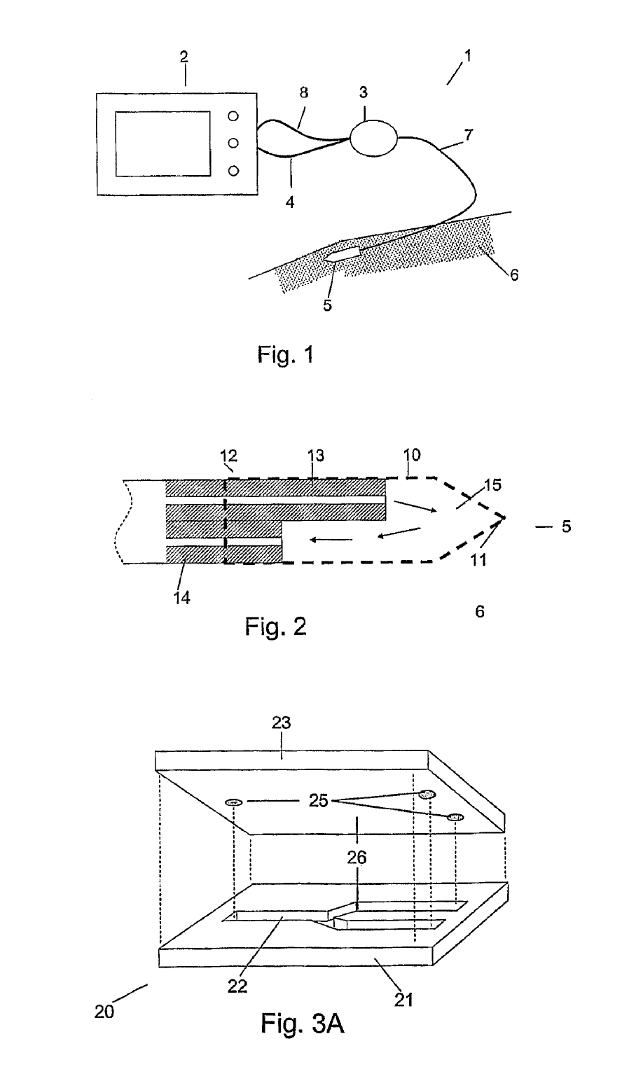

FIG. 1 shows a system (1) for measuring substance of interest in a medium such as a gas or fluid, where the medium in a more specific example is blood or body tissue. In the preferred embodiment of the invention, the system (1) comprises a base station (2) containing an exchangeable fluid part consisting of fluids and optionally pumping means, and an electronics part comprising a computer to store and possibly process data and a display to show the measurements. The system could preferably be attached to the mains or could alternatively, especially in a portable version, contain its own energy source such as batteries or fuel cells.

The base station (2) is in fluidic connection with the analyzing unit (3) through the first fluid communication link (4) preferably being commercial availably flexible tubes of the kind generally used in the field of medical infusion systems. The analyzing unit (3) preferably is attached to e.g. the arm or wrist of a patient under surveillance, possible by a plaster, and contains sensing means for performing an analysis of the concentration of the substance. A substance collecting device (5) for collecting the substance from the tissue (6) is in fluidic connection with the analyzing unit (3) through the second fluid communication link (7). The electrical communication link (8) ensures one or more of the operations of connecting the analyzing unit with an energy source, and communicating measured digital or analogy data from the analyzing unit to or/and from the base station. In a special embodiment of the invention this is replaced by wireless communication.

The fluid communication link (4) comprises a collection of a number of individual tubes (408) in a common coating as it is described later on.

The substance collecting device (5) is any kind of structure able to collect the substance of interest from the medium (6), but is in the preferred embodiment of the invention a commercial available micro dialysis probe, or just probe, of the kind where a perfusion fluid is transported to the first side of a semi-permeable membrane having its second side in contact with the medium (6). As the perfusion fluid flows along the membrane it collects the substance of interest as they diffuse across the membrane from the medium (6), this substance enriched fluid perfusion fluid, now referred to as a sample fluid, is then removed from the membrane for further analysis. A typical example of such a probe is described in WO9413195A1.

FIG. 2 shows a simplified illustration of such a probe (5), where a tube-shaped semi-permeable membrane (10) has its distal end (11) closed and the proximal end (12) attached around the forward fluid conduit (13) and the rearward fluid conduit (14). The perfusion fluid is supplied to the inside (15) of the membrane through the forward fluid conduit, and leaves through the rearward fluid conduit. As the perfusion fluid travels from the forward (13) to the rearward (14) fluid conduit, it collects substance diffusing across the membrane from the surrounding medium (6). Thus, it is a substance enriched perfusion fluid, called sample fluid, leaving through the rearward fluid conduit (14)

The conduits (13, 14) preferably are standard commercial available fluid transport tubes with dimensions in the range of micrometers, like coated glass tubes with inner diameters in the range of 5-50 mm and outer diameters in the range of 500-1000 mm. However, other materials may also apply. The conduits (13, 14) may be arranged either as separate tubes, or where a single tube contains both conduits (13, 14) side by side, or perhaps in a concentric system having one conduit inside the other.

Alternatively to the tube-shaped membrane (10), it may be introduced a window in one the conduits (13, 14).

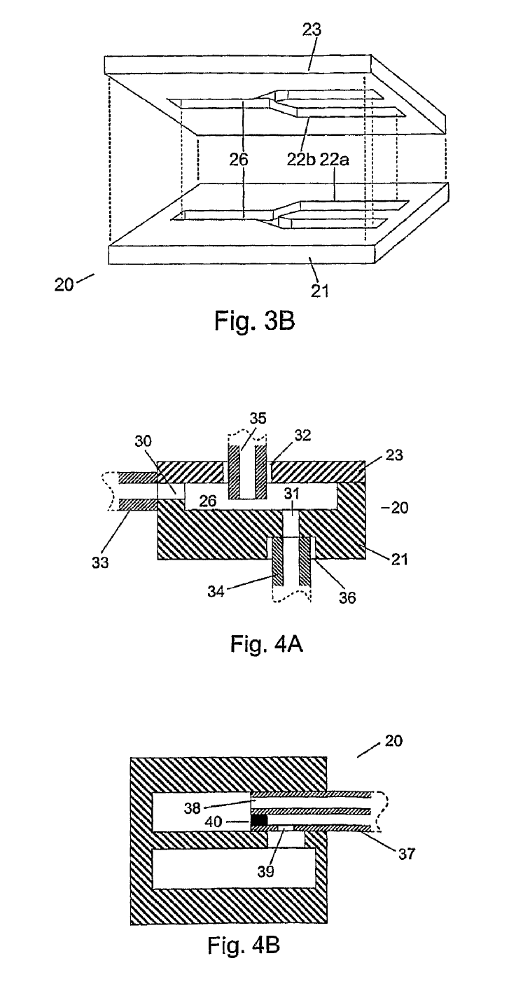

FIG. 3A shows an illustration of a general microfluidic chip (20), where a base plate (21) has grooves in one surface forming flow path(s) (22). Though it is referred to as a plate, any shape applies having at least one surface suitable for forming flow path(s).

The base plate (21) is preferably a substrate in which the flow path(s) is/are formed. It may be made from a polymer material, including, but not limited to, polystyrene (PS), polymethyl methacrylate (PMMA), polyethylene terephtalate glycol (PETG), cyclic olefin copolymer (COC), and/or any other suitable polymer material. Alternatively, the base plate may be made from another suitable material which is not a polymer. The flow path(s) may be formed in the base plate using any suitable kind of technique, such as etching or hot embossing, or the base plate may be manufactured using an injection moulding technique, the flow path(s) in this case being formed in the base plate during the manufacturing process. Alternatively, any other suitable technique known per se in the art may be used for forming the first flow path(s). The flow path(s) will normally be formed in a surface part of the base plate.

The flow path(s) (22) is made fluid-tight by covering the base plate (21) with a top plate (23), where the first top plate could be a plate of dimensions comparable to those of the base plate (21), or a thin foil, and is aligned with the base plate (21), thereby forming the flow path(s) (22) into channels (26). The top plate could be of the same material as the first base plate, or of another suitable material, like the ones described above, and in the preferred embodiment it has a substantially planar and smooth surface, possible having through holes (25) aligning with the flow path(s) (22), creating access from the channels (26) to the environment.

The top plate is preferably attached to the base plate by laser welding, ultrasound welding, heat welding, or any other welding method, but any other method may be applied like gluing or adhering in any known way. It may be attached at the whole surface area of the base plate where there is no flow path(s), or just around the edges of the flow path(s).

FIG. 3B shows a special embodiment of a similar general microfluidic chip (20), where the top plate (23) is a mirrored version of the base plate (21) also comprising flow path(s) (22b), so that when the two plates (21, 23) are connected, the flow path(s) in the top plate align with the flow path(s) (22a) of the base plate (21), the channels formed (26) being partly in the base plate (21) and partly in the top plate (23).

FIG. 4A illustrates a side view of the general chip (20), where the hole (30) is pierced through the side of the base plate (21), the hole (31) pierces the bottom of base plate (21) and the hole (32) as well as the holes (25) on FIG. 3A pierce the top plate (23). In the following any such of the holes (30, 31, 32) creating access from the channels (26) to the environment, piercing the base plate (21) or the top plate (23), or both, are in general just referred to as openings.

Tubes like the ones forming the first fluid communication link (4) are connected to the openings either by pressing them against the top plate (23) or the base plate (23), like the tube (33) being pressed respectively against the side of the base plate (21), aligning the inner flow channel with the opening (30). Some fluid tightening material may also be inserted between the tube and the base plate or the top plate, in order to create a tight fluidic connection from the inside of the tubes to the channels (22). Alternatively the tubes partly or totally penetrate the openings, like the tube (35) penetrating the opening (32) or the tube (34) being inserted into a widening (36) of the opening (31).

FIG. 4B shows a method of attaching a multi-lumen tube (37) to a microfluidic chip (20), seen as a top view with two channels, where the end-opening (38) of at least one lumen is fluidically connected to a first channel(s) of the microfluidic chip (20), and at least one of the other lumens is connected to a second channel(s) through an opening in the side of the tube (37) but is closed at the end-opening, possibly with some plug (40).

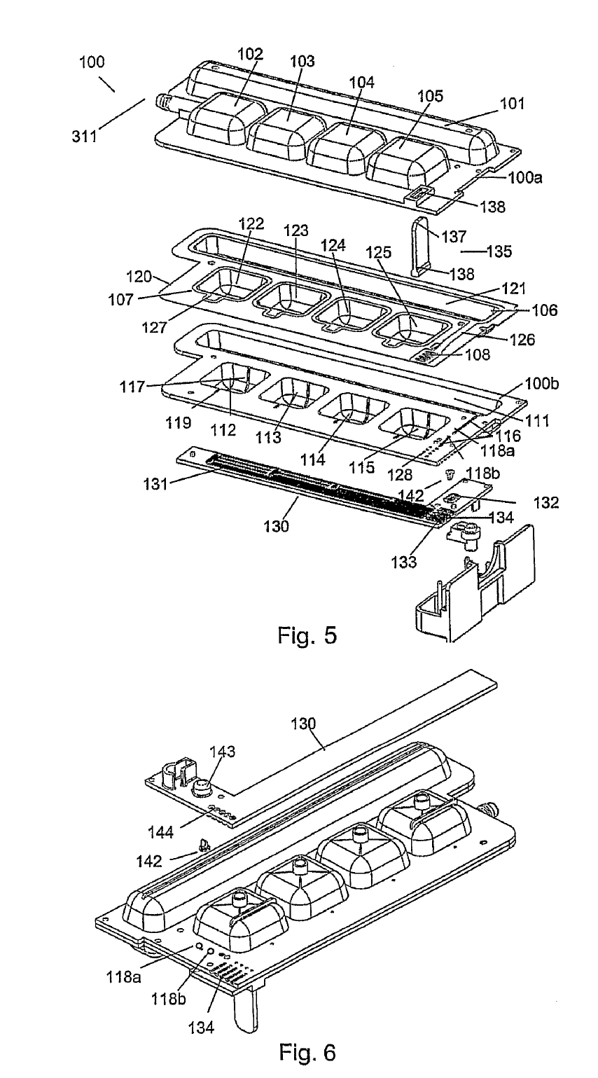

FIG. 5 shows the reservoir system comprising the pressurized structure (100). The main components of the pressurized structure (100) are an upper element (or the pressure side element) (100a), a lower element (or the the fluid side element) (100b), a foil or membrane (120) and a manifold element (130).

The pressure side element (100a) has a first group of shapes or geometries (101-105), and the fluid side element (100b) have a second group of shapes or geometries (111-115), the first and second group of geometries being outward appearances in the plates constituting the bodies (100a, 100b), the first group of geometries of the upper body in size and shape approximately mirroring the second group of geometries of the lower body. The first group geometry (101) is roughly of the same internal volume as the combined internal volumes of the first group geometries (102-105) and correspondingly is the second group geometry (111) roughly of the same internal volume as the combined internal volumes of the second group geometries (112-115).

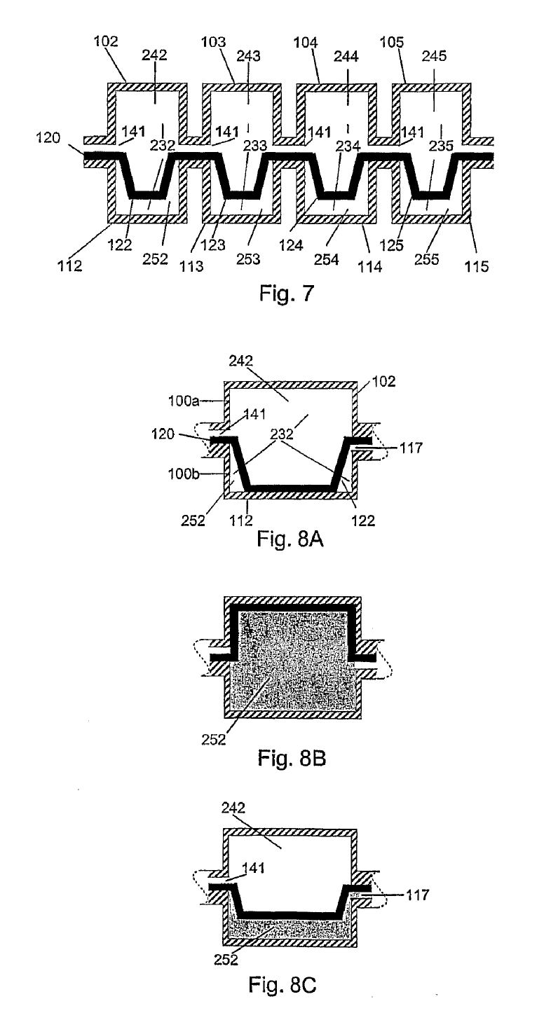

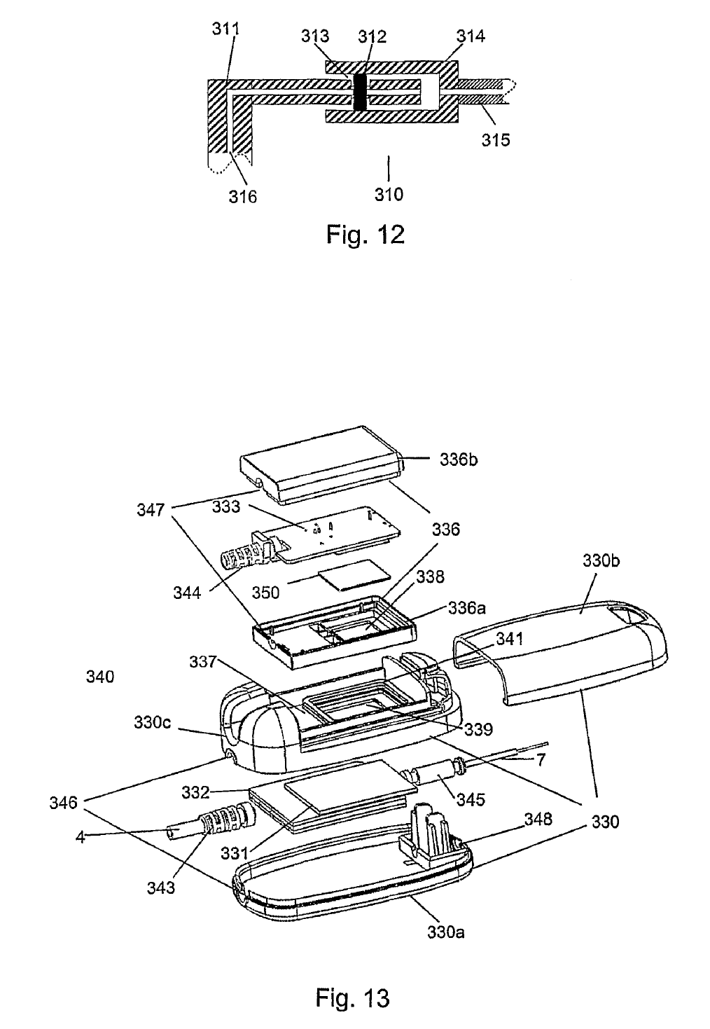

The pressure side element (100a) has internal first pattern of recesses (141, see e.g. FIG. 7) connecting the inside of the first group of geometries (101-105) to the connector (311) of the pump connection (310), seen on FIG. 12. These recesses may be separately connected to the connector (311) but preferably only one of the first group geometries (101-105) is directly connected to the connector (311), the first group geometries being connected to each other by the first pattern of recesses.

A foil or membrane (120) of a flexible material has been positioned between the pressure side element (100a) and the fluid side element (100b), where flexible is understood in the way that the material is capable of easily bending without injury, but without necessarily having any significant elasticity. The membrane (120) has sections (121-125) shaped to fit into the first and second groups of geometries (101-105, 111-115), where the shaping is preferably formed by vacuum moulding and/or punching or stamping.

Alternatively, to ensure that a pressure gradient across the membrane may squeeze the fluids out of the reservoir, the membrane (120) may in another embodiment comprise flexible portions being more elastic than a remainder portion of the membrane, the flexible portions being located between aligned cavities, and where the flexible portions are adapted to be deformed essentially into a shape of at least one of the two cavities located on each side of the flexible portion.

The elements (100b, 120) are preferably connected around the rims (106-108) of the second group of geometries (111-115) and the membrane sections (121-125) by heat welding of the foil or membrane (120) onto the fluid side element (100b). But any other way of connecting elements could also apply, like adhering, laser welding or ultrasound welding.

The pressure side element (100a) is preferably also attached to the membrane (120) at the surface opposite to the one attached to the fluid side element (100b), this is preferably done by ultrasound welding around the rims (106, 107), but any other way of connecting elements could also apply like adhering or laser welding.

Preferably all the fluid side geometries (112-115) are equipped with second pattern of recesses (117) having reservoir through holes (119) penetrating the pressure side element (100b), where at least sections of the second pattern of recesses (117) are aligned with the bulges (127) in the rims (107), and part of the second pattern of recesses (117) are located inside the second group of geometries (112-115), as it is seen on the figure. The second group geometry (111) has a waste recess (116) aligning with the rim bulge (126), where the waste recess (116) has two separate sections, the first section waste recess having a first check valve through hole (118a), and the second section waste recess having a second check valve through hole (118b).

The rim bulge (126) surrounds the waste recess (116), the first and second check valve through holes (118a, 118b), and a number of valve through holes (128) also pierced through the fluid side element (100b), in a way where the rim bulge (126) contains free islands, or connections, (108) of the flexible membrane (120) covering the second through holes (128), each connection (108) covering a pair of the valve through holes (128), in such a way that each of the valve through holes (128) is paired with one other of the valve through holes (128), the connection (108) creating fluid communication between them.

A manifold element (130) is attached to the pressure side element (100b) at the surface opposite to the membrane (120). The manifold element (130) has a first manifold recess pattern (131) and a second manifold recesses (133) formed in the surface, where the first manifold recesses (131) is connected to the reservoir through holes (119) and the valve through holes (128). The recesses (131, 133) may have mirrored recesses in the surface of the fluid side element (100b) forming channels when the manifold element (130) and the fluid side element (100b) are connected. The first and second check valve through holes (118a, 118b) are in fluidic connection with the inside (132) of a check valve geometry (143) wherein a check valve (142) is arranged.

Since the first manifold recess pattern (131) are connected to the valve through hole (128), a fluidic connection between the inside of the second group geometries (112-115) and the second manifold recesses (133) is established. In the same manner the second section of the waste recess (116) is connected to one of the second through holes (128), establishing fluidic connection between the inside of the second pattern geometry (110) and one of the second manifold recesses (133).

The two elements (130, 100b) are preferable attached by ultrasound welding, laser welding, or in any other known way to attach two objects. The first manifold recesses (133) include fluid openings (134) either in the side of the element(s) (100b) and/or (130), as seen on the figure, or through one of the top or bottom surfaces of one of the elements (100b) or (130).

A valve (135) is sited with a first end (136) pressing the connections (108) of the flexible membrane (120) down onto the lower element (100b), thereby blocking any fluidic access through the valve through holes (128). The second end (137) of the valve (135) is situated through the slit (138). The valve (135) is preferably made of some substantially soft material like rubber. When the system is set into operation, the valve (135) is removed, preferably irreversibly, thereby freeing the fluidic access through the valve through holes (128).

The pressure side element (100a), the fluid side element (100b) and the manifold element (130) are preferably equipped with dowels and holes, the dowels fitting into corresponding holes when the elements (100a, 100b) and (100b, 130) are connected, in order to ensure sufficient alignment of the elements, and to give a stable connection.

FIG. 6 shows the manifold element (130) and the pressure side element (100a) connected to the membrane (120) and the fluid side element (100b).

Fluidic communication between the two check valve through holes (118a) and (118b) is achieved through the check valve (142) ensuring that flow can only run from the check valve through hole (118b) to (118a) and not vice versa. The check valve (142) is preferably a standard commercially available duck bill valve, but any other check valve may also apply. The check valve (142) is arranged within a check valve shape or geometry (143) that has the inner (132) in fluidic communication with the check valve through holes (118a, 118b), and the check valve (142) is positioned in the check valve geometry (143) in a manner where fluid may only flow from the check valve through hole (118b) via the check valve (142) to the check valve through hole (118b).

If tubes like the individual tubes (408, see e.g. FIG. 15) are to be inserted into e.g. the recesses (133), then optionally through holes (144) may be introduced where through glue or other adhesion materials may be filled to fix tubes in the recesses.

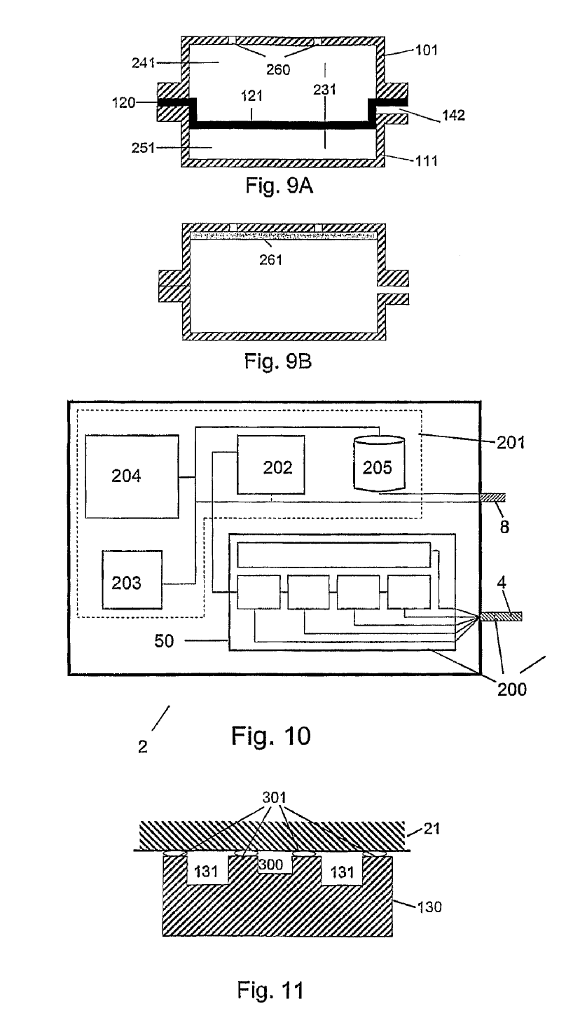

FIG. 7 shows a side view illustration of the combined system of the pressure side element (100a), the fluid element (100b) and the membrane (120). When the elements (100a, 100b) are connected, the first group of geometries (101-105) align with the second group geometries (111-115) forming the compartments (231-235) (compartment (231) is seen on FIGS. 9A and 9B). The membrane sections (122-125) separate the compartments (232-235) into the upper (or pressure) chambers (242-245) and the lower (or fluid) chambers (252-255), and the membrane sections (122-125) seal the pressure chambers (242-245) from the fluid chambers (252-255) in a manner that is tight to gas or liquid.

The fluids are fed to the internal of the fluid chambers (252-255) through accesses (not shown), where the accesses are openings into the first group of geometries (102-105) giving fluid connection to the environment. The accesses are preferably equipped with lure lock taps for attaching the means transferring the fluids to the fluid chambers (252-255). Subsequently the accesses are closed, preferably by melting the lure lock taps by heat welding, or they are sealed in any other way of closing such openings.

FIG. 8A is a side view illustrating one of the compartments (232) formed by the first group geometry (102) and second group geometry (112), the figure also showing the first and second pattern of recesses (117, 141). The flexible membrane (120) is squeezed between the two elements (100a, 100b) so that the membrane section (122) separates the compartment (232) into two chambers (242) and (252), as described above, each chamber having an access to the environment, the chamber (242) through the first pattern of recesses (141) in the surface of the pressure side element (100a), and the chamber (252) through the second pattern of recesses (117) in the surface of the fluid side element (100b), as also described above.

In FIG. 8B the compartment (232) is shown when it is filled with a fluid, the fluid being inside the fluid chamber (252) defined by the membrane section (122) and the shape (112). A gas like air, or some fluid, is feed into the pressure chamber (242) through the first pattern of recesses (141), creating a pressure gradient rise across the membrane section (122), said pressure gradient ensuring that the fluid inside the fluid chamber (252) is squeezed through the second pattern of recesses (117).

FIG. 8C shows the same compartment (232) where a large amount of the fluid has been squeezed out of the fluid chamber (252), as gas or liquid has been filled into the pressure chamber (242).

FIG. 9A shows the waste compartment (231) for storing the waste fluid(s), designed in a similar manner to the compartments (232-235) of the first group geometry (101) and the second group geometry (111), the waste compartment is split into the two chambers, the waste chamber (241) and the dummy chamber (251), by the membrane section (121). Preferably the waste fluid is led into the waste chamber (251) through the waste recess (116) as in the illustrated embodiment of the invention, however, the opposite situation may also apply. The dummy chamber (241) is then just a ‘dummy’, making no significant counter pressure on the membrane section (121).

The dummy chamber (241) is ensured to be under a lower internal pressure than the waste chamber (251), preferably by having free access to the external atmospheric pressure through the atmospheric holes (260) in the first group geometry (101). In this embodiment, the membrane section (121) ensures that no fluid present in the waste chamber (251) leaks to the environment through the atmospheric holes (260), since it seals the waste chamber (251) from the dummy chamber (241). Alternatively the membrane section (121) could be avoided by sealing any such atmospheric hole (260) with a fluid tight but air permeable membrane(s) (261), as it is shown in FIG. 9B.

In a special embodiment of the invention, the ‘dummy’ chamber would operate to regulate the pressure in the system, thereby regulating the flow rates. This might be realized by pressurizing the second chamber in some way. The check valve (142) ensures that no back flow runs from the waste chamber (251) back into the second manifold recesses (133).

FIG. 10 shows a simple illustration of the base station (2) comprising the pressurized structure (100), the pumping means (202), the electronics (201), monitoring means (204) and optionally the energy resources (205), like a battery or fuel cells. In the preferred embodiment of the invention however, the energy is obtained from the mains.

The exchangeable fluid part, also called the wet parts, (200) constitutes the pressurized structure (100), the analyzing unit (3), the probe (5), and the first and second fluidic communication link (4) and (8), but could in specific embodiments of the invention also include especially the pumping means (202) and/or the energy resources (205).

The electronics (201) is foremost a computer (203) and a monitoring device (204). The computer is mainly for storing and processing the measurement data, but could additionally take on other possible tasks like storing the set up information of the system and information about the object of surveillance, e.g. a patient. The monitoring device (204) is preferably a standard monitor possibly having a touch screen.

The base station further comprises whatever electronics and mechanical devices known to a person skilled in the art of electric devices to be needed in such an electrical apparatus.

The pumping means is preferably a compressor pumping air or some other gas or liquid into the pressure chambers (242-245) at some adjustable constant rate and pressure. Any other imaginable way of pumping would also apply to the system, like a mechanical or electrical system squeezing a fluid (gas or liquid) out of a flexible container into the pressure chambers (242-245).

The wet parts (200) is attached to or inserted into the base station (2) in any way know to a person skilled in the art, like placing it into a cavity inside the enclosure (or box) of the base station (2), where the cavity is shaped to contain the connected upper element (100a) and lower element (100b) in a fixed and stable manner.

One important aspect of the present system, is that it offers the possibility, that e.g. a patient equipped with the wet parts (200) may be transferred between several base stations (2), where the wet parts (200) is inserted, and the surveillance either started or continued. When moved from one location to another, the patient would not need to feel the discomfort of having the probe (5) removed and a new one inserted, but could keep the same probe (5) and the rest of the wet parts (200).The wet parts (200) may advantageously also comprise means for storing such data as the already obtained measurements and/or the set up information of the system, where the means advantageously could be a digital microchip. Alternatively such data could be transferred directly wirelessly between the individual base stations.

Another main aspect of the present invention is that no parts of the wet parts (200) comprises metal, or at least so small amounts of metal, that they do not interfere with e.g. an MRI-scan.

FIG. 11 shows a feature especially for the manifold element (130), but it would also apply to the pressure side and fluid side elements (100a, 100b). The manifold element (130) is welded to the fluid side element (100b) in such a way, that any two adjacent first manifold recesses (131) are separated by a double welding, since this offers extra safety in the case that one welding should not be fluid tight. Then there is still a chance that the second welding would still prevent fluids in the first manifold recesses (131) to intermix.

A further safety feature is introduced in the preferred embodiment of the system as it is also shown on the figure, where drain channels (300) are formed along the side of the first manifold recesses (131). The figure shows a cross section of the manifold element (130) showing two of the first manifold recesses (131) having a drain channel (300) between them, the lower element (100b) being welded (301) to the manifold element (130) in the areas between the channels (131) and the drain channel(s) (300), or attached in some other manner.

The drain channel(s) (300) ensures that a fluid leaking through an un-tight welding would be ‘captured’ by the drain channel (300) and removed before it could leak into an adjacent first manifold recess (131) in an observable manner, possibly by letting it drain out of the system to the environment. Thereby a leak appearing in the manifold element (130) could be detected, and the defect wet parts (200) replaced.

FIG. 12 shows the pump connection (310) to the wet parts (200), where air is the preferred pressurizing substance to be feed into the pressure chambers (242-245), but any gas or liquid may also apply. The pump connection (310) is shown as a two-part system where the male connector (311) is equipped with an O-ring (312) fixed in a rift (313) in a way where part of the O-ring is above the rift (313) and has a diameter just a little larger than the inner diameter of the female connector (314). When the male connector (311) is positioned inside the female connector (314) the friction of the O-ring against the inner wall of the female connector (314) ensures a sufficiently stable and fluid-tight connection, thereby establishing a fluid connection between the pressure tube (315) and the pressure inlet (316), the air inlet being in fluidic communication with the recesses (141) and there through the pressure chambers (242-245) seen on FIGS. 8A-8C. The male connector (311) could be an integrated part of one of the elements (100a, 100b, 130), but is preferably a separate part attached to the pressure element (100a).

It is an advantage to keep the means for analyzing close to the patient or what the medium under investigation is, where the means for analyzing comprises the analyzing unit (3), second fluid communication link (7) and substance collecting device (5). This is due to the need to minimize the response times of the system during measurement, and due to the general patient comfort, where only this analyzing part is attached to the patient, the freedom of movement being limited only by the length of the first fluid communication link (4) and the electrical communication link (8).

FIG. 13 shows the preferred embodiment of the design of the analyzing unit, where the analyzing unit casing (330) is made of three parts, the analyzing unit casing bottom (330a), the analyzing unit casing top (330b), and the analyzing unit support structure (330c). The system operates by the principle of adding reagents to a sample fluid comprising the substance of interest, to give some detectable effect, thus the analyzing unit comprises an analysis microfluidic chip (331), also called a micro lab, where the reagent fluids are mixed to the sample fluid to give the observable and measurable effects representative of the concentration of some substance in a fluid, where the effects in the preferred embodiment are optically detectable.

A manifold microfluidic chip (332) distributes the fluids in the system, such as feeding reagent fluids to the analysis microfluidic chip (331) and optionally distributing waste fluid(s) away from the analyzing unit. In a preferred embodiment the manifold microfluidic chip is also feeding perfusion fluid to the inward conduit (13) of the substance collecting device (5), and receiving the sample fluid from the rearward fluid conduit (14) distributing it to the analysis microfluid chip (331).

The analyzing unit support structure (330c) is placed between the analyzing unit casing top (330b) and the analyzing unit casing bottom (330a) and is shaped with a deepening (337). A sensor casing (336) comprises a sensor casing bottom (336a) and a sensor casing top (336b), and is positioned in the deepening (337). The surface of the analyzing unit support structure (330c) opposite to the inside of the deepening (337) presses the fluidic parts (331) and (332) against the analyzing unit casing bottom (330a) keeping it fixed, advantageously using a substantially soft member (possible a rubber washer, rubber gasket or foam) placed between the fluid parts (331, 332), the analyzing unit support structure (330c) and/or the analyzing unit casing bottom (330a).

Both the analyzing unit support structure (330c) and the sensor casing bottom (336a) are equipped with windows, a first window (339) in the analyzing unit support structure (330c), and a second window (338) in the sensor casing bottom (336a), the two windows positioned to align when the pieces of the analyzing unit are put together. A washer, gasket or foam (341), preferably of rubber or elastomere, is compressed between the analyzing unit support structure (330c) and the sensor casing bottom (336a) around the windows (338, 339) to seal against especially external light sources. Thereby the sensor (333) enclosed between the sensor casing bottom (336a) and sensor casing top (336b) only receives light entering through the windows (338, 339). A transparent sheet or plate (350) may be positioned in the window.

The three analyzing unit casing parts (330a, 330b, 330c) are preferably connected along the rims by ultrasound welding, but any other method also applies, like adhering the parts. In the same way the two sensor casing parts (336a, 336b) are preferably connected along the rims by ultrasound welding, but any other method also applies, like adhering the parts.

The measurement of the substance is as described above in the preferred embodiment based on optical detection, so it is critical that no external light sources enter either the microfluidic chips (331, 332) or the inside of the sensor casing (336) where the sensor is positioned. Therefore the casing (330) is designed so that the only fluidic connection from the environment to the internal fluidic parts (331, 332) is through the first fluid communication link (4). The only other connection to the environment is the electrically conductive communication link (8) connecting the sensor (333) to the base station (2). The first and second fluid communication link (4, 7) and the electrical communication link (8) are equipped with plugs (343, 344, 345), respectively, for sealing the accesses (346, 347, 348).

One of the main parts of the analyzing unit is a microfluidic chip performing the chemical reactions giving the observable and measurable optic effects representative of the concentration of some substance in a fluid.

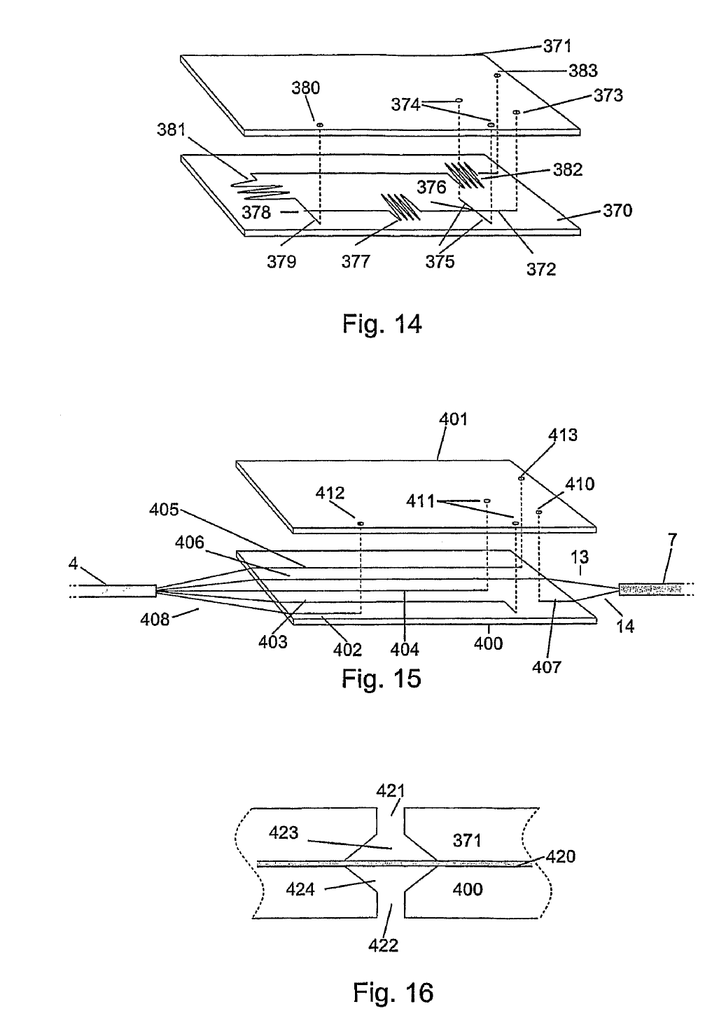

FIG. 14 shows a preferred design of the analysis microfluidic chip (331) designed in the same way as the general microfluidic chip (20) of an analysis base plate (370) and an analysis top plate (371) having an analysis channel system (372, 375, 377, 379, 381, 382) enclosed between them. In the figure the two parts (370, 371) are not yet connected. The perfusion fluid enters the analysis channel (372) through the analysis chip opening (373) in the analysis top plate (371). A first reagent fluid enters the analysis channel section(s) (375) through the analysis chip opening(s) (374) in the analysis top plate (371) and is merged with the perfusion fluid at a mixing point (376). The analysis channel (372) continues into a first meandering section (377) where the merged fluids get time to intermix and react before they reach the second mixing point (378) and merge with a second reagent fluid entering from the analysis channel section(s) (379) and analysis chip opening(s) (380). The analysis channel (372) continues into a second meandering section (381) where the merged fluids get time to intermix and react before they reach the third meandered section (382), which is aligned with the windows (339, 338), so that the sensor (333) has a view to the optically detectable reactions occurring at the third meandered section. The first top plate (371) therefore has to be transparent at least where it coverers this third meander. The fluid, now a waste fluid, leaves the analysis microfluidic chip (331) through the analysis opening (383).

FIG. 15 shows a preferred embodiment of the manifold microfluidic chip (332) designed in the same way as the general microfluidic chip (20) of a manifold base plate (400) and a manifold top plate (401), the two plates (400, 401) not yet connected at the figure. A number of manifold channels (402-407) are in fluidic connection with the individual tubes (408). The manifold channels (402-407) are further in fluidic connection with a set of manifold chip openings (411-413). In the illustrated embodiment of the invention one of the manifold channels (406) connects one of the individual tubes (408) to forward fluid conduit (13) of the substance collecting device (5), the one communicating the perfusion fluid. Optionally the perfusion fluid bypasses the analyzing unit all together, one of the tubes (408) being directly connected to the forward fluid conduit (13).

The manifold channel (407) in the illustrated example creates fluidic connection between the rearward fluid conduit (14) of the substance collecting device (5) and the analysis channel (372) in the analysis microfluidic chip (331) through the manifold chip opening (410) and analysis chip opening(373).

The manifold channels (402-407) are preferably made by flow path(s) in the manifold base part (400) aligned with mirrored flow path(s) in the manifold top plate (401) as it was described above.

Alternatively the forward fluid conduit (13) of the substance collecting device (5) and/or the rearward fluid conduit (14) are directly connected to the analysis microfluidic chip (331), where the manifold microfluidic chip (332) distributes the perfusion and the sample fluid to and from the analysis microfluidic chip (331).

The two microfluidic chips (331, 332) are attached together with the filter (420, see FIG. 16) positioned between them, in such a way that the openings align to create fluid communication between the channels of the two microfluidic chips. The example system at the figure then has the manifold chip opening (410) aligning with the analysis chip opening (373), the manifold chip openings (411) align with the analysis chip openings (374), the manifold chip opening (412) aligns with the analysis chip opening (380) and the manifold chip opening (413) aligns with the analysis chip opening (383).

The filter may be positioned alternatively, such as defined by the direction of flow, right before the manifold microfluidic chip (332) or perhaps positioned right after the second manifold recesses (133), preferably it just has to be inserted before the flow restrictors (501), where the flow restrictors (501) are described below.

The two microfluidic chips are preferably connected in a base-to-top manner, where the analysis base plate (370) is positioned against the manifold top plate (401), but alternatively it could be base-to-base, where the analysis and manifold base plates (370, 400) are positioned against each other, top-to-top, where the analysis and manifold top plates (371, 401) are positioned against each other, or top-to-base where the analysis top plate (371) is positioned against the manifold base plate (400).

The two microfluidic chips (331, 332) are connected by ultrasonic welding, heat welding, gluing or by any other way of connecting two elements.

FIG. 16 shows one connection of two openings (421, 422), where the manifold base plate (400) is positioned against the analysis top plate (371), and where filter recesses or depressions (423, 424) are formed around the openings (421, 422) and are aligned. The filter (420) is positioned between the filter depressions (423, 424) and the two microfluidic chips (331, 332) for removing dirt, pollution, microbes and other materials possibly being present in the fluids. The two filter depressions (423, 424) increase the operation area of the filter. The filter material is preferably a PES of millipores, but any suitable filter may also apply.

A preferred way of regulating the individual flow rates of the system, is to introduce flow restrictors or flow restricting elements into the system, where the flow restrictors advantageously could be tube sections having a significantly smaller inner cross-sectional area than the individual tubes (408), the first and second manifold recesses (131-133) and the manifold chip and analysis chip channels (402-407, 372, 375, 377-379, 381, 382) of the manifold microfluidic chip (132) and the analysis microfluidic chip (131). A natural choice for such flow restricting elements would be a portion of a standard commercially available silicon based micro bore tube, or capillary tube, the capillary tube having the property that for any given pressure difference the flow rate may be fixed at a desired value by choosing a capillary of suitable length and diameter.

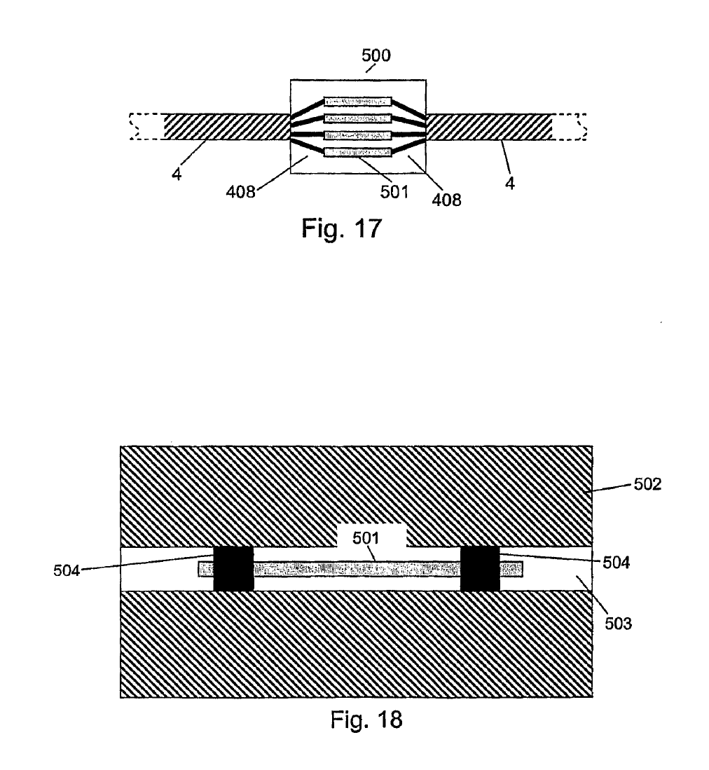

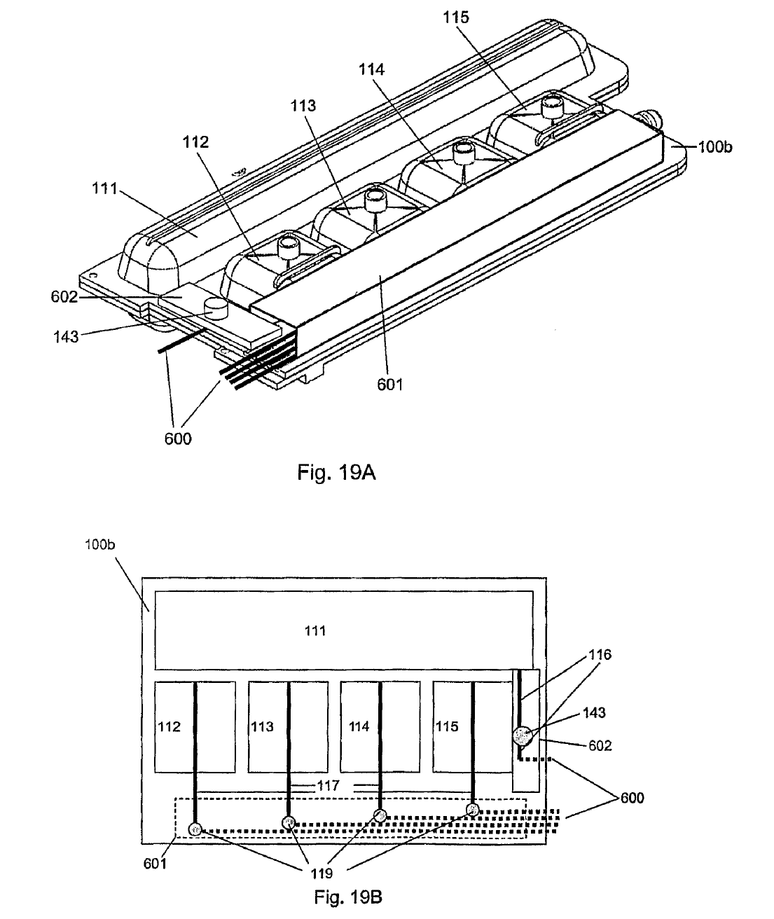

A number of different embodiments of inserting the flow restrictors are possible, like introducing them into the first or second manifold recesses (131-133), or, as it is shown on FIG. 17, by introducing a restriction collection (500) at the first fluid communication link (4), where a flow restrictor (501) is inserted for each of the individual tubes (408) increasing the total flow resistance of the individual tubes (408), thereby lowering the flow rates. The preferred embodiment of the invention is, however, to insert the flow restrictors (501) into the channels of either the manifold microfluidic chip (332) or the analysis microfluidic chip (331), as it is seen on FIG. 18 showing a top view of a microfluidic chip (502) and a channel (503), where a flow restrictor (501) is positioned in the channel (503) and fixed by one or more plugs (504) of some adhering material, also working to seal against the fluid flowing in the channel forcing it though the flow restrictor (501).

The system in the preferred embodiment operates in such a way that a perfusion fluid is transported from a container in the base station (2) through one of the individual tubes (408) in the first fluidic communication link (4) to the forward fluid conduit (13) of the probe (5), optionally passing through one of the microfluidic chips (331) or (332). In the probe (5) substance of interest are collected by the sample fluid as they diffuse across the membrane (10). This enriched perfusion fluid, now a sample fluid, enters the meandering first channel(s) (372) of the analysis microfluidic chip (331), as it was described previously, optionally first passing through one of the channels of the manifold microfluidic chip (332).

The sample fluid is subsequently mixed with a number of reagent fluids. The reagent fluids are transported from containers in the base station (2) through some of the individual tubes (408) in the first fluidic communication link (4), entering the analysis microfluidic chip through openings like (374, 380) and channel sections like (375, 377), and mixed with the sample fluid at mixing points like (376, 378). Meandering reaction sections (377, 381) ensure that the reaction fluids have time to mix sufficiently with the sample fluid and react.

The final meandering section (382) is where the detectable optical effects are detected by the sensor (333) through the window (338). The sensor sends the measured data to the base station (2), preferably through the electrical communication link (8) or by a wireless transmission.

The sample fluid, now a waste fluid, leaves the analysis microfluidic chip (331) through an opening (383), preferably passing through the manifold microfluidic chip (332) and one of the tubes of the fluid communication link (4), back to the base station (2), where it is stored in some bag, container or chamber (231). Alternatively the waste fluid is led directly out of the system.

The base station (2) comprises a computer being able to process and store the data, and preferably a monitor for displaying them. The computing device (2) may also be capable of controlling the sensor (333) and/or the flow rates in some way.

The flows of the fluids are created by pumping means (202) contained in the base station (2), where the pumping means in the preferred embodiment is of the kind where the fluids are stored in fluid bags or chambers (252-255), each having at least one flexible side or wall in pressure communication with one of the pressure chambers (232-235). Alternatively all the fluid chambers (252-255) are positioned in one common pressure chamber. The internal part of the pressure chamber is then filled with a pressurizing substance like a gas, whereby the fluids inside the fluid chambers are squeezed into the fluid communication link (4) as the pressurizing substance pushes the flexible side or wall against the fluid.

The first fluid communication link (4) is preferably a number of individual flexible tubes (408, see FIG. 15) as they are known in the field of medical infusion systems, and preferably made of materials like PE, PUR, PA, etc. The individual tubes (408) are assembled in a common coating or outer jacket, made of materials like PVC, Rubber, PUR, etc. In order to ensure correct mounting of the tubes, especially during assembly of the system, the individual tubes would preferably be coloured differently, perhaps also having different outer diameters, to ensure that only one mounting permutation of the tubes is possible.

FIG. 19A shows an alternative version of the pressurized structure e.g. seen at FIGS. 5 and 6, where the manifold element (130) is replaced by tubes (600) and a protective casing (601) protecting the tubes (600) and optionally fixing them to the pressure side element (100b). The tubes replace at least the first and second manifold recesses (131, 133) and are fluidic connected to the reservoir through holes (119). In the preferred and illustrated embodiment the tubes (600) are just the individual flexible tubes (408) extending from the common coating to the reservoir through holes (119).

A valve casing element (602) comprising the check valve geometry (143) is in this embodiment attached to the pressure side element (100b). The system of the valve casing element (602) and pressure side element (100b) may have recesses (116, 133), openings (134) and through holes (118) like the system of the embodiment seen in FIGS. 5 and 6, and operates in the same manner.

FIG. 19B illustrates the same system as FIG. 19A just seen from the top, where the dashed lines illustrate the back side of the system with the tubes (600) and protective casing (601). The top front side of the illustration comprises the second group geometry (111-115), the first pattern of recesses (117) creating fluidic communication from the inside of the second group geometry (112-115) through the reservoir through holes (119) to the tubes (600), the waste recess (116) and the valve casing element (602) with the check valve geometry (143).

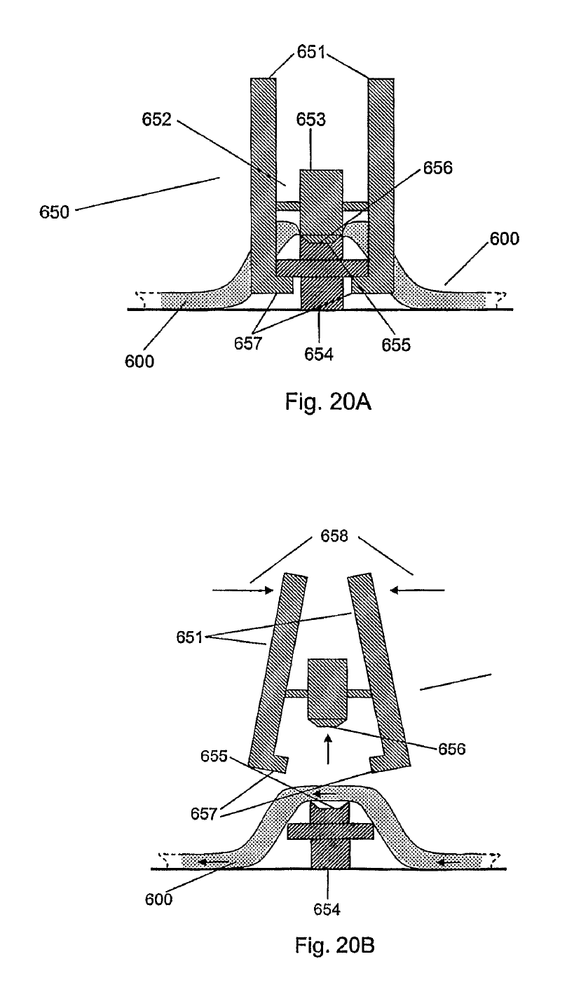

FIGS. 20A and 20B shows an alternative version of the valve (135) especially suited for the embodiment seen at FIGS. 19A and B. A clamping element (650) comprising two legs (651) and a pivot element (652) comprising a pressing element (653), is releasable attached to a structure (654) positioned where the tubes (600) are free from the protective casing (601). The structure (654) is positioned under the tubes and comprises a mating section (655) to receive and fit an end portion (656) of the pressing element (653). Each of the two legs (651) comprises a part (657) to secure the clamping element (650) to the structure (654), and when the clamping element (650) is secured to the structure (654) the tubes (600) are squeezed between the end portion (656) and the mating section (655) to deny any fluid communication through the tubes (600). When the system is to be set into operation, the two legs (651) are pressed together around the pivot element (652) as illustrated by the arrows (658) at FIG. 20B, to free them from the structure (653), and the climbing element (650) is then removed thereby freeing fluid communication in the tubes (600).

While the present invention has been illustrated and described with respect to a particular embodiment thereof, it should be appreciated by those of ordinary skill in the art that various modifications to this invention may be made without departing from the spirit and scope of the present invention.

What is claimed is:

1. A system for analysing a fluid, the system comprising a base station, an analysing unit being remote from the base station, and a substance collecting device being remote both from the base station and from the analysing unit, a first fluid communication link for communicating fluid between the base station and the analysing unit, and a second fluid communication link for communicating fluid between the analysing unit and the substance collecting device, wherein the analysing unit comprises sensing means adapted to provide data representing a content of a substance in the fluid and wherein the base station comprises data processing means being adapted to process the data to provide information regarding the content of the substance in the fluid.

2. The system according to claim 1, wherein the substance collecting device comprises a membrane.

3. The system according to claim 1, wherein the information relates to a physiological condition of a living being.

4. The system according to claim 3, wherein the sensing means comprises a sensor and is adapted for optically sensing of a characteristic of the fluid.

5. The system according to claim 1, wherein the first fluid communication link comprises separated flow channels for communication of different fluid separate from each other.

6. The system according to claim 1, wherein the base station and the analysing unit are adapted to exchange data via a data exchange link.

7. The system according to claim 6, wherein the data exchange link comprises an electrical cable connection,

8. The system according to claim 1, wherein the first fluid communication link comprises a detachably attached tubular element.

9. The system according to claim 7, wherein the tubular element and electrical cable connection is comprised in one common link between the base station and the remote sampling station.

10. The system according to claim 1, wherein the first fluid communication link is different from the second fluid communication link.

11. The system according to claim 10, wherein the first fluid communication link is longer than the second fluid communication link.

12. The system according to claim 10, wherein the first fluid communication link has smaller flow resistance than the second fluid communication link.

13. The system according to claim 1, wherein the second fluid communication link comprises a forward fluid conduit for conduction of fluid from the analysing unit to the substance collecting device, and a rearward fluid conduit from the substance collecting device to the analysing unit.

14. The system according to claim 13, wherein the first fluid communication link comprises a primary fluid conduit which is in direct fluid communication with the forward fluid conduit.

15. The system according to claim 1, wherein base station comprises a computing device and a reservoir system, the analysing unit and the reservoir system being detachable from the computing device.

16. The system according to claim 15, wherein the base station further comprises pumping means being detachable from the reservoir system and from the analysing unit.

17. The system according to claim 15, wherein the analysing unit is in electrically communication with the computing device.

18. The system according to claim 6, wherein the reservoir system comprises at least one flexible reservoir in pressure communication with a pressure chamber.

19. The system according to claim 18, wherein at least one of the flexible reservoirs comprises a perfusion fluid suitable for sampling of substance is based on micro dialysis, the reservoir being in fluid communication with the substance collecting device.

20. The system according to claim 18, wherein the at least one flexible reservoir is separated from the pressure chamber by a flexible membrane, the pressure chamber being in fluidic connection with the pumping means.

21. The system according to claim 1, wherein the analysing unit comprises an optical sensor arranged in optical communication with a microfluidic chip.

22. The system according to claim 1, wherein at least the analysing unit and the substance collecting device is made entirely of material(s) compatible to a MRI scan.
